(19) United States
(12) Patent Application Publication
(10) Pub. No.: US 2025/0084134 A1
(43) Pub. Date: Mar. 13, 2025

(54) KERATIN HYDROLYSIS PEPTIDE SOLUTION

(71) Applicant: CH Biotech R&D Co., Ltd., Nantou City (TW)

(72) Inventors: Kai XIA, Nantou (TW); Ching-Chi TSAI, Taichung City (TW); Meng-Ying LI, Nantou (TW); Yu-Lun LIU, Taichung (TW); Nai-Hua YE, Changhua (TW)

(73) Assignee: CH Biotech R&D Co., Ltd., Nantou City (TW)

(21) Appl. No.: 18/527,787

(22) Filed: Dec. 4, 2023

(30) Foreign Application Priority Data
Sep. 8, 2023 (TW) 112134393

(51) Int. Cl. C07K 14/465; C07K 1/12; C07K 1/34; G01N 33/68

(52) U.S. Cl. CPC C07K 14/465; C07K 1/12; C07K 1/34; G01N 33/6848; G01N 2333/465

(57) ABSTRACT

Present invention uses a mixture of feathers and water, and, depending on specific embodiments of the ratio between feathers and water, subject the mixture to a high-temperature and high-pressure treatment process for a fixed amount of time, resulting in a keratin solution containing at least 253 peptides, with their molecular masses between 500 and 4,000 Daltons, and the concentration is in the range of 2.0×10⁵˜4.5×10⁵ ppm. The solution can then be further diluted by water for application to different plants, at specific growth stages, following additional steps or processes of application to increase crops yield.

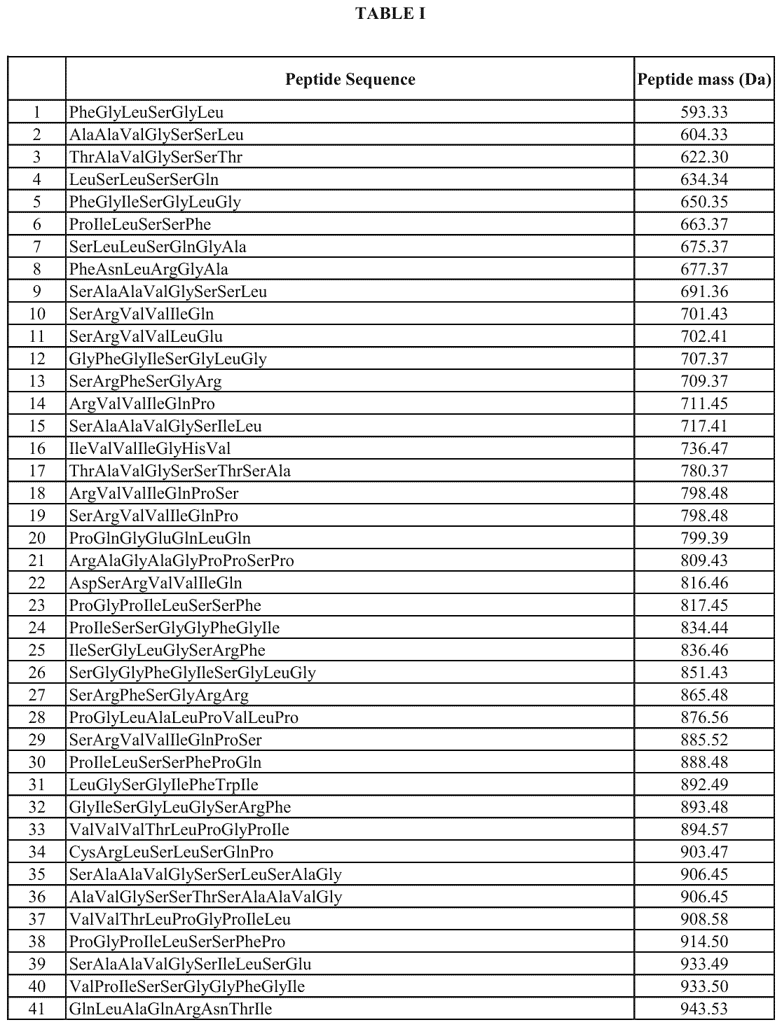

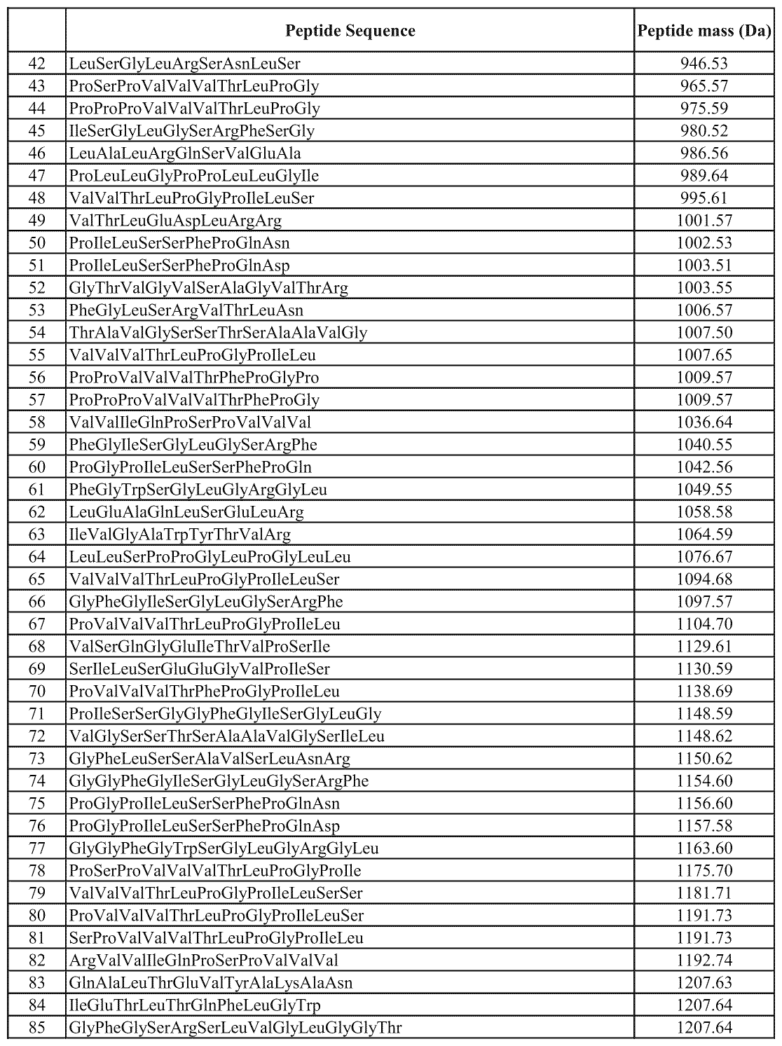

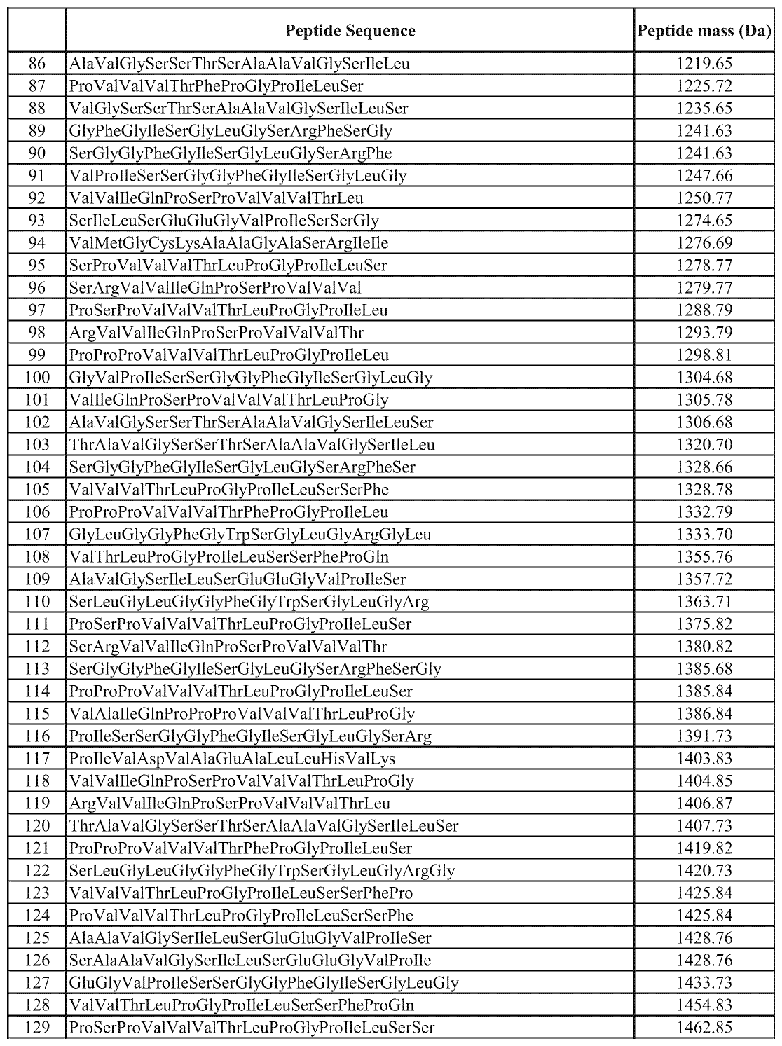

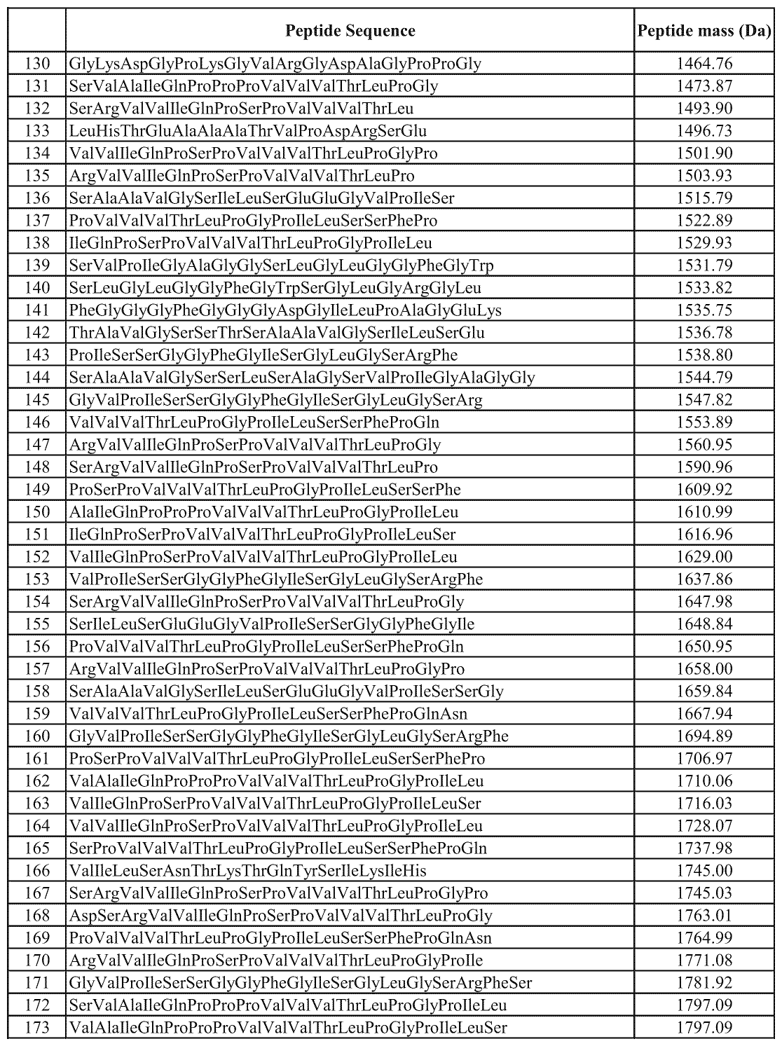

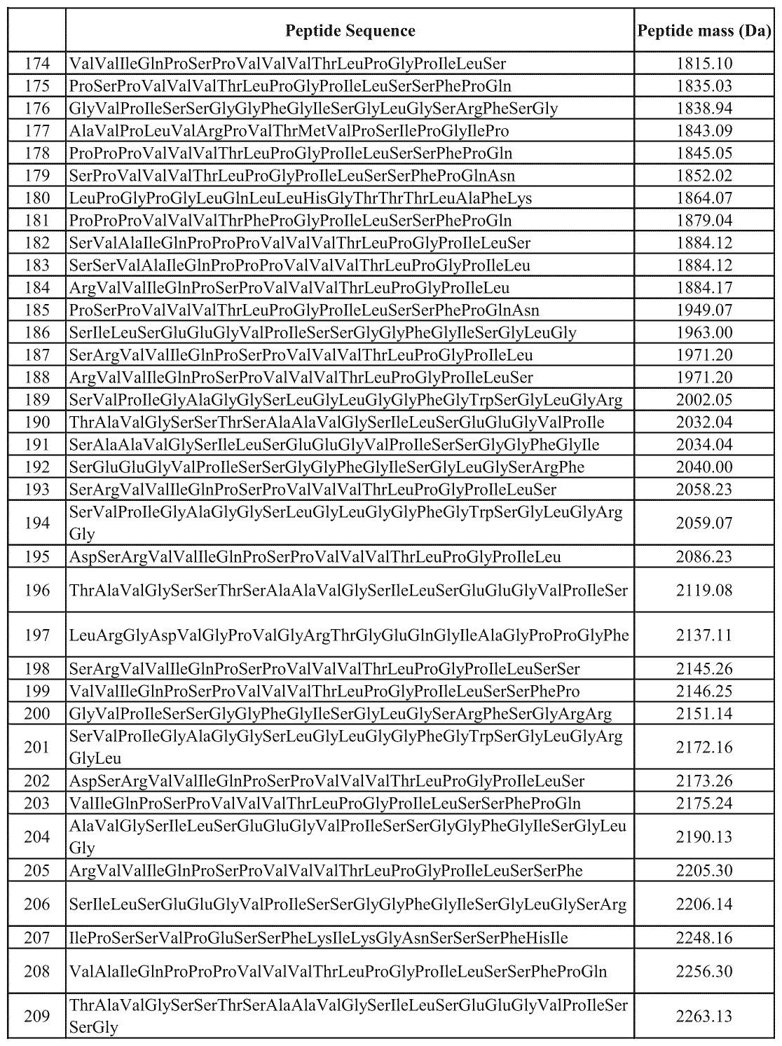

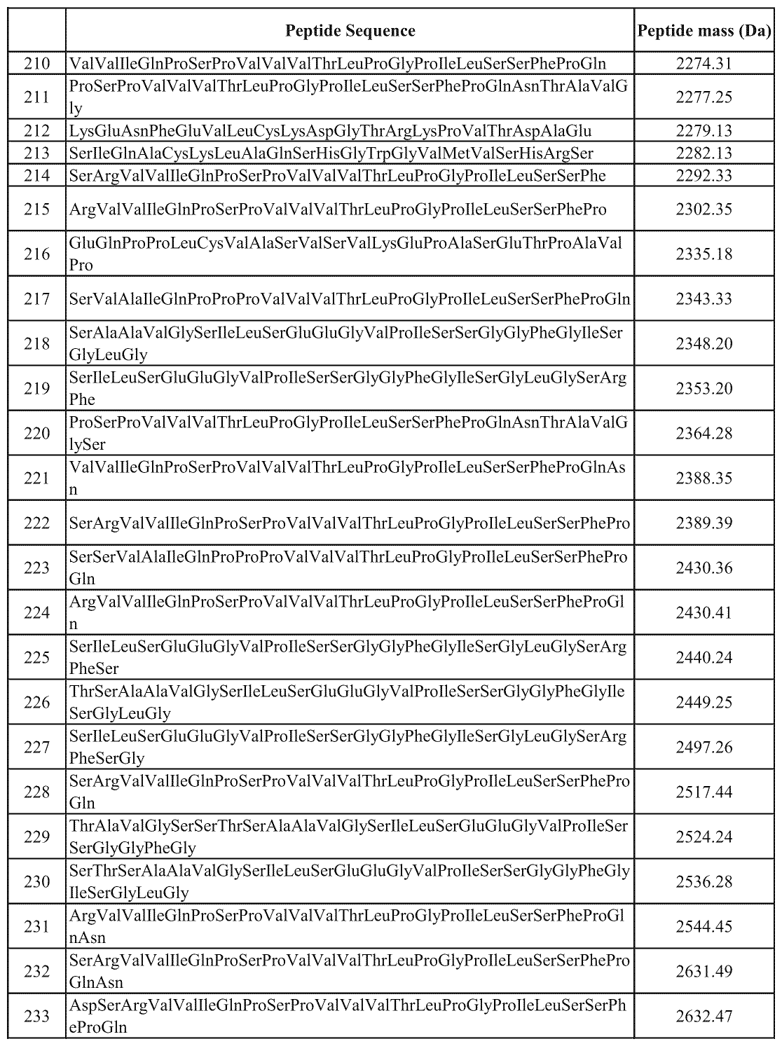

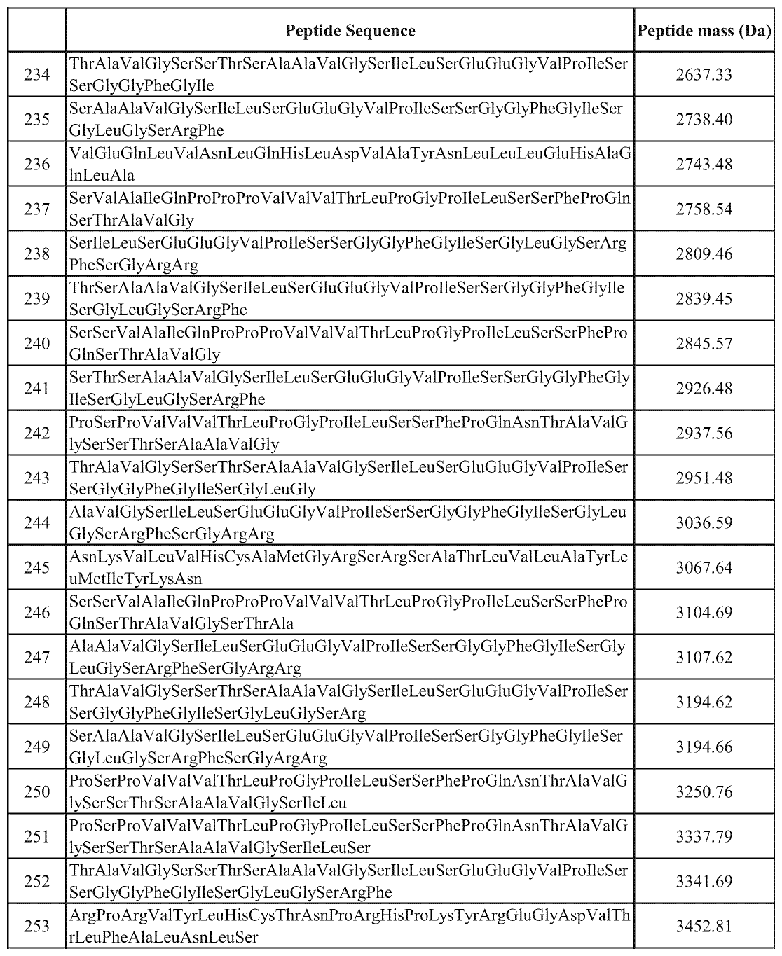

### PRIORITY CLAIM TO FOREIGN APPLICATION

Applicant hereby makes priority claim to a Taiwan patent application, number 112134393, having the Taiwan filing date of Sep. 8, 2023.

### BACKGROUND OF THE INVENTION

Present invention disclosed and claimed a keratin hydrolysis peptide (“KHP”) solution that is made via a high-temperature and high-pressure process, resulting in a solution that has many beneficial applications in the fields of horticulture, agriculture and potentially other farming businesses.

Hydrolyzed keratin has long been used to strengthen hairs, reduce breakage, and minimize damages. Some form of chemical process is used to break down the large protein molecule of the hydrolyzed keratin so that it can penetrate the hair cuticles and do its magic.

The inventors of present application experimented and discovered that by employing a specific high-temperature and high-pressure hydrolysis process to break down the keratin and to form a hydrolyzed peptide solution, it has great beneficial uses in agricultural and horticultural fields.

The KHP solution made by the components in the amount stated herein and treated by the specific process disclosed can be used to increase coffee production yields, grape production yields, enhance the flavors of coffee and grape, strength the drought resistance feature of certain crops, as well as many other plants when applied under specifically regimented methods.

### SUMMARY OF THE INVENTION

It is well known that feathers of domestic birds/fowls contain 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by mixing feathers and water (generally at a rate of 1:3) and the going through a high-temperature and high-pressure (setting of 160° C., 6.1 kg/cm², or setting of 180° C. and 9.2 kg/cm² for a duration of 30 minutes) to obtain a solution.

The keratin solution is then further mixed with other fertilizers containing nitrogen, potassium and other minerals that can be applied to some plants in the hope of increasing the production yield of the plants.

The Nurdiawati keratin solution, however, has few empirical tests to confirm its beneficial uses, and to what plants.

The inventors of present application experimented and found out certain mixture of feathers and water can be treated by a high-temperature and high-pressure process, and will result in a keratin solution that is shown to be beneficial in agricultural purpose.

The invention uses a mixture of water and feathers (water content 50%) at the weight ratio of 2:3 and subject the mixture to a thermal hydrolysis process (40 minutes) to create a solution, resulting in the solution having molecular mass of 593.3-3,828.0 Dalton and containing at least 253 peptides. Some of the bioactive peptides were confirmed by referencing the BIOPEP-UWM database.

The solution can be further filtered and concentrated to 200,000 ppm.

A second embodiment uses the water and feathers (water content 50%) of 4:5 and then treat it to a thermal hydrolysis process (80 minutes), resulting in a solution with molecular mass of 593.3-3,508.9 Dalton, containing at least 253 peptides. Some of the bioactive peptides were confirmed by the BIOPEP-UWM database.

A third embodiment uses feathers only, with 46% water content, and treat it with the same thermal hydrolysis process (40 minutes), resulting in a solution with molecular mass of 705.9-3194.7 Dalton, containing at least 253 peptides. Some of the active bioactive peptides were confirmed by the BIOPEP-UWM database.

### BRIEF DESCRIPTION OF DRAWINGS

The accompanying drawings, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

### DETAILED DESCRIPTION OF THE INVENTION

The keratin hydrolysis peptide (“KHP”) solution of present invention, as a first specific embodiment, is made by a high-temperature and high-pressure process to treat a mixture of water and feathers where the weight ratio whereby the weight ratio of water and feathers is set at 2:3.

The specific weights used for the water and feathers in the mixture consists of mixing 44 kg of water and 66 kg of feathers, with the water content of the feathers being 50%.

The mixture then is treated by the steps of:
a. stirring the feathers and water mixture in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 593.3 and 3,828.0 Daltons.

The keratin hydrolysis peptide (KHP) solution obtained by the elements and process described above further filtered and concentrated to 200,000 ppm concentration.

A second embodiment for the water and feathers mixture (again, the feathers are of 50% water content) is set to have a weight ratio of 4:5.

A specific embodiment for mixture of second embodiment consists of mixing 40 kg of water and 50 kg of feathers, and then is treated by the steps of:
a. stirring the feathers and water mixture in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification whereby their molecular masses are between 593.3 and 3,508.9 Dalton.

The keratin hydrolysis peptide (KHP) solution of second embodiment it is further filtered and concentrated to 301,500 ppm concentration.

A third embodiment of keratin hydrolysis peptide (KHP) solution, without water, can be made by 70 kg of feathers, with the feathers' water content being 46%, and then treated by the steps of:
a. stirring the feathers in a sealed container;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 705.9 and 3,194.7 Dalton.

The keratin hydrolysis peptide (KHP) solution of the third embodiment is further filtered and concentrated to 381,250 ppm concentration.

Generally, the molecular masses of the selected peptides are between 500 and 4,000 Daltons. The selected peptides will have between 5-30 amino acids.

The at least 253 peptides obtained and confirmed via the BIOPEP-UWM database are identified herein as shown in Table I in the specification.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The step of further filtration and concentration can be done via a stainless steel bag filter housings, such as one made by Taiwan Grace International Corp with additional air compressor to increase pressure and to speed up the filtration pace.

As disclosed herein, the keratin hydrolysis peptide (KHP) solution thus obtained, with further researches and experiments, can be further diluted by water before using for specific plants/crops.

Additional applications to different plants for various agricultural and/or horticultural fields as may be desired by users of present invention, where additional steps or processes are followed that will help with the increase of yield or production of crops or enhancement the drought-resistance of said crops/plants.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A keratin hydrolysis peptide (KHP) solution comprising:
water and
feathers of 50% water content mixed into the water
whereby the weight ratio of water and feathers mixture is set at 2:3 and is then treated by the steps of:
a. stirring the feathers and water mixture in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 593.3 and 3,828.0 Daltons.

2. The keratin hydrolysis peptide (KHP) solution of claim 1 whereby the mixture by weight of 2:3 is done by specifically mixing 44 kg of water and 66 kg of feathers.

3. The keratin hydrolysis peptide (KHP) solution of claim 2 is further filtered and concentrated to 200,000 ppm concentration.

4. A keratin hydrolysis peptide (KHP) solution comprising:
water, and
feathers of 50% water content mixed into the water
whereby the weight ratio of water and feathers mixture is set at 4:5 and is then treated by the steps of:
a. stirring the feathers and water mixture in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification whereby their molecular masses are between 593.3 and 3,508.9 Dalton.

5. The keratin hydrolysis peptide (KHP) solution of claim 4 whereby the mixture by weight of 4:5 is done by specifically mixing 40 kg of water and 50 kg of feathers.

6. The keratin hydrolysis peptide (KHP) solution of claim 5 is further filtered and concentrated to 301,500 ppm concentration.

7. A keratin hydrolysis peptide (KHP) solution comprising:
70 kg of feathers, with the feathers' water content being 46%, and treated by the steps of:
a. stirring the feathers in a sealed container;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 705.9 and 3,194.7 Dalton.

8. The keratin hydrolysis peptide (KHP) solution of claim 7 where it is further filtered and concentrated to 381,250 ppm concentration.
